(19) United States
(12) Patent Application Publication
(10) Pub. No.: US 2024/0221915 A1
(43) Pub. Date: Jul. 4, 2024

(54) SYSTEMS AND METHODS FOR A HEALTHCARE PORTAL

(71) Applicant: SAVVOS, American Fork, UT (US)

(72) Inventors: Jacob FACKRELL, American Fork, UT (US); Lucien Morin, American Fork, UT (US); Michael Jensen, American Fork, UT (US)

(73) Assignee: SAVVOS, American Fork, UT (US)

(21) Appl. No.: 18/090,388

(22) Filed: Dec. 28, 2022

(51) Int. Cl. G16H 40/20; G06Q 20/08

(52) U.S. Cl. CPC G16H 40/20; G06Q 20/085

(57) ABSTRACT

Methods, systems, and devices for arranging, completing, and paying for healthcare services using a portal. The systems and methods include: (1) requesting a healthcare procedure, (2) scheduling the healthcare procedure, (3) confirming pricing for the healthcare procedure, (4) authorizing payment for the healthcare procedure, (5) funding the healthcare procedure, (6) completing the healthcare procedure, (7) paying for the healthcare procedure, and/or (8) communicating with the healthcare provider, the payor, and/or the facility using the portal.

### BACKGROUND

Healthcare services typically involve a plurality of healthcare providers (doctors, nurses, etc.) providing services at a plurality of facilities (hospitals, doctors' offices, etc.) paid for by a plurality of payors (insurance companies, patients, etc.). Finding healthcare providers that can provide the necessary healthcare services can be confusing, cumbersome, and time consuming. Additionally, at least some healthcare providers do not publish prices for their services, making comparing prices of healthcare providers nearly impossible. Moreover, ensuring that payors will pay for the healthcare services needed from the chosen provider at the chosen facility can also be confusing, cumbersome, and time consuming. In essence, in the current healthcare system, a patient typically has to be their own project manager, which can be difficult for patients who are not familiar with the healthcare system.

Moreover, consumers may face difficult obstacles when attempting to request a “price” for a medical procedure. They may spend hours on the phone trying to connect with someone at a medical facility, only to be told it's not possible to share a price for care upfront, or they may be referred to their health plan or their physician. The ability for consumers to know the price of care, before receiving care, is complex, time consuming, and incredibly frustrating. The current model in healthcare is to let a third party, such as a health plan, determine how much a procedure will cost, and not share this information with a member/patient.

Furthermore, this problem is compounded by the constraints placed upon medical providers. Many times, these providers are part of insurance networks or other contractual pricing relationships that are so complex the provider doesn't really know how much they will receive for care. In addition, many medical providers are prohibited from sharing their contractual pricing with others.

Accordingly, a need exists for a system that streamlines the process of discovery, access, scheduling, payment, and delivery of healthcare to patients. Such improvements may take advantage of advancements in computer systems and computer-related technologies.

### SUMMARY

The described technologies include improved methods, systems, devices, or apparatuses that provide, among other things, improvements to how healthcare services are arranged, completed, and paid for.

The systems and methods described herein create a consumer facing solution that allows the patient to search for a common medical procedure and identify medical providers who perform this procedure, and the estimated “advertised” price for this procedure. This allows patients to identify multiple providers that may be a suitable solution for them and identify useful measurables such as price, ratings, distance, quality or credentialed surgeons.

The ability for patients to access this information results from medical providers across the country creating a basic account within the systems described herein. This allows medical providers access to their provider portal to list common procedures by description or CPT code, and to list a price associated with that procedure. When these procedures and prices are added to the provider's portal, this information is organized by the systems described herein and displayed in search results when members/patients are searching for these medical procedures.

Medical providers list their best “cash price” for services within the system described herein. These prices can be changed at any time by the medical provider, and those changes are effective immediately within the system described herein. Medical providers can also list their own price for the procedure or can list a “bundled price” that includes other participating providers. For example, a surgical center may list a price for an ACL Repair and may also include the price charged by the surgeon, the price charged by the anesthesiologist, the price charged by the physical therapist, and more. Providers have the flexibility to list prices as best fits their practice and operations.

The system described herein also links the medical provider to a messaging center, allowing providers to communicate with a patient when that provider is selected by the patient. The patient can communicate directly with the provider through the messaging center to ask questions, to schedule care, to finalize a price for care, and to approve the cost of the procedure before receiving care.

As such, one aspect of the present disclosure relates to methods, systems, and devices for arranging, completing, and paying for healthcare services using a portal that creates a nationwide cash-pay marketplace for healthcare services. The systems and methods include: (1) requesting a healthcare procedure, (2) scheduling the healthcare procedure, (3) confirming pricing for the healthcare procedure, (4) authorizing payment for the healthcare procedure, (5) funding the healthcare procedure, (6) completing the healthcare procedure, (7) paying for the healthcare procedure, and/or (8) communicating with the healthcare provider, the payor, and/or the facility using the portal.

### BRIEF DESCRIPTION OF THE DRAWINGS

A further understanding of the nature and advantages of the present invention may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 illustrates an example of a system that supports arranging, completing, and paying for healthcare services using a portal in accordance with aspects of the present disclosure.

FIG. 2 illustrates an example of a system architecture that supports arranging, completing, and paying for healthcare services using a portal in accordance with aspects of the present disclosure.

FIGS. 3-7 show flowcharts illustrating methods that support arranging, completing, and paying for healthcare services using a portal, and other aspects of the present disclosure.

FIG. 8 shows a diagram of a system including a device that supports arranging, completing, and paying for healthcare services using a portal, and other aspects of the present disclosure.

### DETAILED DESCRIPTION

The systems and methods disclosed herein relate to, among other things, the discovery, accessibility, arrangement, payment, and delivery of healthcare to patients. Arrangement of healthcare services may be a cumbersome and frustrating process. The systems described herein enable a patient to search for, arrange, schedule, and pay for healthcare services by enabling the patient, the healthcare providers, and the payors to communicate in a single portal that streamlines the process of delivering healthcare to the patient. As such, the systems described herein may substantially eliminate the patient's effort to obtain, schedule, and pay for healthcare services.

One aspect relates to a system that uses a single portal that is streamlined and easy to use. The portal creates a single interface where patients, healthcare providers, facilities, and payors can communicate and facilitate healthcare services. The portal includes a marketplace module, an instant messaging center, a scheduling module, and a payment module in a single portal that enables patients, healthcare providers, facilities, and payors to communicate directly with each other to facilitate the delivery, scheduling, and payment of healthcare services.

Specifically, the portal includes a technology platform to support a national marketplace to enable healthcare providers to: (1) advertise cash prices for specific healthcare services that are made available to health plans and their members/patients; (2) connect healthcare providers, health plans, and members/patients in a single conversation to negotiate final cash prices for requested healthcare services; and (3) guarantee and deliver immediate payment to providers by health plans and members/patients upon the completion of healthcare services.

Additionally, healthcare providers create an account within the portal and add all physical locations to the marketplace. Healthcare providers may then advertise services within the portal by adding Current Procedural Terminology (CPT) codes with a corresponding cash price to each physical location. These listed procedures are organized in search results based on CPT code, procedure description, location, and search radius. Search results display the cash price for a facility and procedure and may also include additional cash prices for professional or other fees such as surgeon, anesthesiologist, therapy or hardware.

Healthcare providers set a cash price for each procedure or healthcare service and list that cash price within the marketplace. Healthcare providers may also update cash prices at any time through the portal.

Additionally, the portal includes payors (insurance companies, employers, third party administrators, health sharing communities, and/or other healthcare payors) who are engaged in the effort of reducing medical costs for their insureds, employer groups, and/or members/patients. Specifically, the portal connects payors with the marketplace to: (1) connect payor membership with cash prices when shopping for healthcare services; (2) improve the healthcare service through a single conversation within the messaging center that connects all interested parties; (3) allow healthcare providers, payor members/patients, and payors to review and approve the final cost of healthcare services in advance of performance of the healthcare service; and (4) reduce or eliminate complex billing issues for the member/patient, the healthcare provider, and the payor by funding the healthcare service through the portal in advance of the scheduled healthcare service to ensure healthcare providers are paid immediately.

The marketplace may be made available only to active payor plans and their members/patients and may not be made available to the general public or offered directly to consumers. Support staff with these payor plans have the ability to search for healthcare services within the marketplace on behalf of their members/patients, or members/patients can be given permissions to perform searches themselves. In alternative embodiments, the marketplace may be made available to the general public or offered directly to consumers.

The portal is configured to execute the methods described herein to enable the discovery, accessibility, arrangement, payment, and delivery of healthcare services to patients. The methods described herein include: (1) requesting a healthcare procedure, (2) scheduling the healthcare procedure, (3) confirming pricing for the healthcare procedure, (4) authorizing payment for the healthcare procedure, (5) funding the healthcare procedure, (6) completing the healthcare procedure, (7) paying for the healthcare procedure, and/or (8) communicating with the healthcare provider, the payor, and/or the facility using the portal.

Requesting the healthcare procedure may include: (1) initiating a search request for healthcare services, (2) selecting a healthcare provider from the search results; and (3) accessing active procedure requests, by the healthcare provider, the payor, and/or the facility, within a dashboard for the healthcare provider, the payor, and/or the facility within the portal. Specifically, representatives of the payors and/or members/patients log into the portal to initiate a search request for healthcare services. The search criteria may include a CPT code or procedure description, as well as a zip code and mile radius. The search results will display a cash price for the healthcare service and/or the facility and may also include other costs associated with the procedure, including the surgeon, anesthesiologist, physical therapist, and/or hardware and supplies.

When the representatives of the payors and/or members/patients select a healthcare provider from the search results, the procedure request begins and prompts the user to share additional information about the member/patient, the member's/patient's physician, the procedure being requested, and the timeline for the procedure. The representatives of the payors and/or members/patients can access active procedure requests within a dashboard. Once the representatives of the payors and/or members/patients log into the portal, access is granted to engage with healthcare providers to proceed with a procedure request. The member/patient must first sign a “Consent and Release” document before proceeding with the procedure request.

Scheduling the healthcare procedure may include: (1) digitally connecting the payor, the patient, and any participating healthcare providers; and (2) proposing a procedure date, by the healthcare provider, based on the information shared by the patient or the payor. The portal provides a digital connection between the payor, the patient, and any participating healthcare providers. Healthcare providers can include the facility (surgical center), the physician (surgeon), and other requested providers (physical therapist, anesthesiologist, etc.). Healthcare providers can be added to a specific procedure request by selecting from a list of active providers within the portal. Once added, the healthcare provider is notified of their addition to a procedure request. The portal also enables the healthcare provider to propose a procedure date, based on the information shared by the patient or the payor. This proposed procedure date can be accepted or declined by the patient. If declined, the healthcare provider will review the comments provided by the patient and propose an alternative procedure date for approval.

Confirming pricing may include: (1) sharing information through an instant messaging center; and (2) finalizing and/or adjusting the price for the requested procedure based on the information shared within the instant messaging center. Once all stakeholders are connected within the portal, information is shared and communicated through the instant messaging center. This allows all participating stakeholders to confirm what care is being delivered, share the necessary documentation, ensure the requested procedure is a covered benefit by the payor, and any other necessary conversations. Healthcare providers may finalize their price for the requested procedure, or to make any necessary adjustments to the price based on the information shared within the instant messaging center. Healthcare providers may build an unlimited number of unique combinations of procedure codes or participating providers in order to create a custom bundled price for the medical procedure request.

The systems described herein enable the stakeholders to create a complete “bundled” price for a medical procedure through an unlimited combination of CPT codes or unlimited combination of independent providers. The ability for providers to offer “cash prices” is complicated by the bundling factor. When a patient needs a medical procedure, there are typically multiple medical providers involved in the care being delivered, and each medical provider has a price that needs to be paid. When a patient needs a “cash price” for a medical procedure they need to work with a group of medical providers that have already “bundled” their prices together, or the patient needs to get a price from each of the providers to know the total cost.

For example, if a patient needs imaging work, such as an MRI, there is the facility cost for the MRI machine, and there is also the radiologist cost for reading the MRI and providing their assessment. In another example, if a patient needs a surgery, such as a hernia repair, there is the cost of the surgical center, the cost of the surgeon, and the cost of the anesthesiologist. When a patient needs a “cash price” for a medical procedure, it can be very difficult for medical providers to create a simple “bundled” price for the patient. Many times the different surgeons and anesthesia groups that work with a surgical center cannot agree on how much they want to charge, they all charge a different price, so it's very complicated to offer a “bundled” price to a patient. In addition, if a group of medical providers can agree on their prices and they offer “bundled” pricing, that price is typically based on a single CPT code, such as 29881 for an ACL Repair. However, many times the patient has additional work that needs to be performed during the surgery, additional CPT codes, so the bundled price needs to be adjusted to account for these changes.

The systems described herein solves these problems by connecting the patient with any unique combination for medical providers, such as a surgical center, a surgeon, and an anesthesiologist. These providers are linked together within a single Procedure Request within the system to allow all providers to engage in a single conversation to determine the needs of the patients. In addition, each provider can finalize their own price within the Procedure Request, creating the ability to build a custom “bundled” price at any time, with any unique combination of providers. The technology platform created by the system described herein allows for flexibility not offered by any other digital healthcare platform.

In addition to linking together providers to create a unique “bundled” price for patients, the system allows each provider to add to, or take away from, the services being offered. For example, if it is determined that a patient needs both CPT code 29881 and 29888, each provider can add additional procedures and prices to the bundle to ensure the unique circumstances of the patient are being met. The system allows for an unlimited combination of CPT codes to create an accurate, final price for medical care that is accessible to the patient before care is delivered.

Authorizing payment for the healthcare procedure may include: (1) withdrawing all necessary funds to cover the costs for the medical procedure being paid for by the payor from the payor's account; (2) authorizing the portal to withdraw funds from the patient's account; and (3) withdrawing all necessary funds to cover the costs for the medical procedure being paid for by the patient from the patient's account. The portal is a financial platform that ensures that healthcare providers are paid immediately and simplifies the payment process by patients and payors. The payor authorizes the portal to pull from the payor's bank account all necessary funds to cover the costs for the medical procedure being paid for by the payor. This financial transaction requires the payor to link an active bank account to the portal and to have funds available for ACH withdrawal up to 5 business days prior to the scheduled procedure date. The patient authorizes the portal to collect any costs for the medical procedure that are the responsibility of the patient, including deductibles or other cost sharing obligations, or for services that are not covered by the payor. This financial transaction requires the patient to link an active bank account or credit card to the portal and to have funds available for withdrawal up to 5 business days prior to the scheduled procedure date.

Providers share a price in the system based on the assurance of receiving immediate payment for services provided. Most providers must wait months to receive payment from a third party payor, such as a health plan, or they battle with patients attempting to collect payment directly. The uncertainty of receiving payment for services provided, and not knowing how much will be received, is one of the main reasons healthcare costs are so expensive. Providers engage in “Revenue Cycle Management” issues to try and maximize the collection of receivables and ensure they get paid.

The system eliminates the need for Revenue Cycle Management for providers by ensuring that providers are paid immediately when care is delivered. As previously noted, the provider portal allows medical providers to share a Cash Price, the Marketplace allows members find this price, the system allows for a single conversation between all parties to schedule care and finalize the pricing, and finally the system ensures that all medical providers are paid immediately.

Within the system, the price of the medical procedure is approved by the patient and by the health plan. Once the price is approved, the health plan and the patient authorize payment for the medical procedure by indicating “Payment Authorized” within the portal. Once payment is authorized, the system pulls the funds from the health plan, and any payment responsibility is also pulled from the member, and these funds are deposited into a clearing account with the system. Once these funds are pulled the procedure is “Funded” and medical providers know that funds are available for payment and will be paid to them immediately when care is delivered.

Once care has been completed, the medical providers mark the procedure “complete” within the portal and funds are released immediately. Payment is made via ACH direct deposit into the providers bank account, unless the provider chooses to receive payment by another method. When medical providers offer care to patients but don't have a contractual relationship with their insurance company or other third party payor, the situation creates tremendous uncertainty for these providers. Not knowing how to ensure that payment will be made to them by the third party payor is the main hurdle in offering a competitive price to these patients. The system not only solves this problem by funding the procedure in advance, but also allows providers to connect with patients that are tied to many different third party payors, and these medical providers don't have to figure out how to work with any of them.

Funding the healthcare procedure may include depositing all funds from the payor and the patient 5 business days prior to the scheduled procedure date. Once the funds are deposited into a portal clearing account, the portal shows the Procedure Request as “Funded”. Healthcare providers can confirm the procedure is funded prior to delivering medical care to verify that payment will be made upon the completion of the medical procedure.

Completing the healthcare procedure may include marking the procedure as complete within the portal. Once a procedure request has been completed, the healthcare provider will mark the procedure complete within the Portal. This step indicates the healthcare provider has delivered the agreed upon care and is ready to receive the agreed upon payment.

Paying for the healthcare procedure may include releasing payment to all providers immediately upon completion of a medical procedure. The portal sends payment via ACH to every healthcare provider that provided an independent price as part of the finalized price within the procedure request.

Communicating with the healthcare provider, the payor, and/or the facility using the portal may include facilitating communication between the healthcare provider, the payor, the patient, and/or the facility using an instant messaging center.

Many other aspects and details associated with the present disclosure are described with reference to the figures as described below. Reference to customer and user may be used interchangeably with patient or other terms identifying a person or living being that provides input into, is observed by, or is otherwise engaged with or focused on using the system and methods disclosed herein.

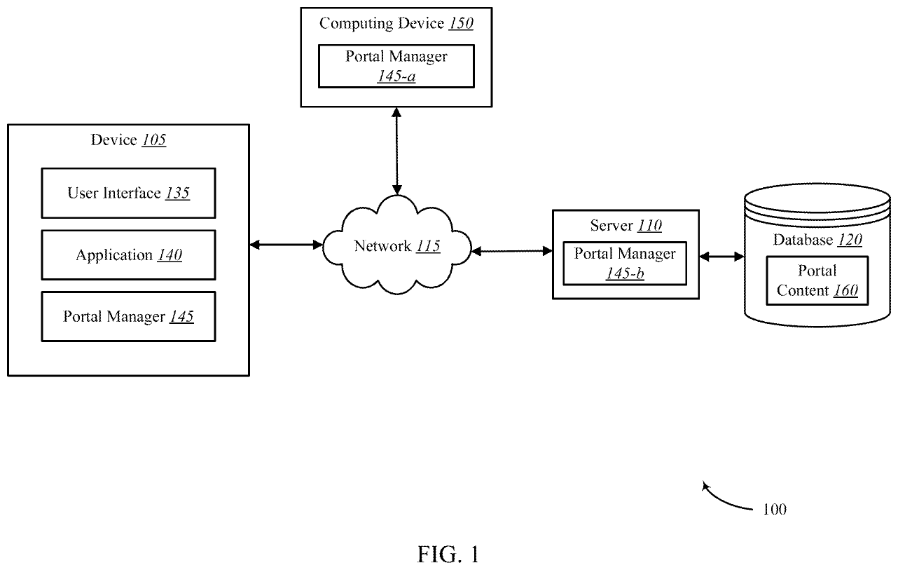

FIG. 1 illustrates a block diagram of one example of a portal system 100 in which the present systems and methods may be implemented. In some examples, the systems and methods described herein may be performed on a device (e.g., device 105). As depicted, the portal system 100 may include a device 105, server 110, a network 115, and a computing device 150, and that allows the device 105, the server 110, and the database 120 to communicate with one another.

Examples of the device 105 may include any combination of, for example, mobile devices, smart phones, personal computing devices, computers, laptops, desktops, servers, media content set top boxes, or any combination thereof.

Examples of computing device 150 may include at least one of one or more client machines, one or more mobile computing devices, one or more laptops, one or more desktops, one or more servers, one or more media set top boxes, or any combination thereof.

Examples of server 110 may include, for example, a data server, a cloud server, proxy server, mail server, web server, application server, database server, communications server, file server, home server, mobile server, name server, or any combination thereof.

Although database 120 is depicted as connecting to device 105 via network 115, in some examples, device 105 may connect directly to database 120. In some examples, device 105 may connect or attach to at least one of database 120 or server 110 via a wired or wireless connection, or both. In some examples, device 105 may attach to any combination of a port, socket, and slot of a separate computing device or server 110.

In some configurations, the device 105 may include a user interface 135, an application 140, and a portal manager 145. Although the components of the device 105 are depicted as being internal to the device 105, it is understood that one or more of the components may be external to the device 105 and connect to device 105 through wired or wireless connections, or both. Examples of application 140 may include a web browser, a software application, a desktop application, a mobile application, etc. In some examples, application 140 may be installed on a computing device in order to allow a user to interface with a function of device 105, server 110, computing device 150, and portal manager 145.

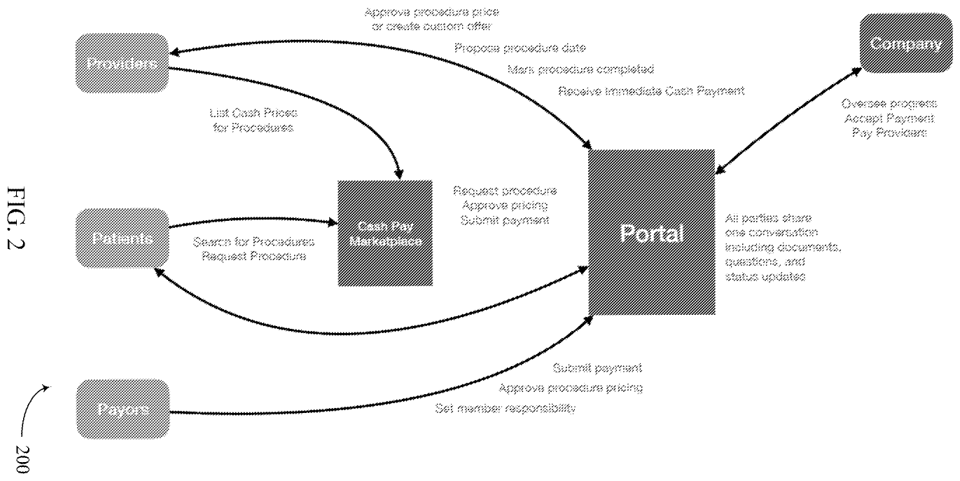

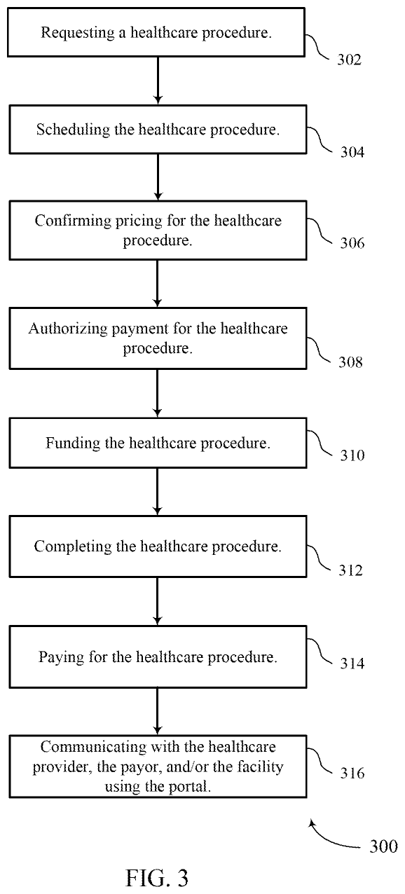

Although device 105 is illustrated with an exemplary single application 140, in some examples application 140 may represent two or more different applications installed on, running on, or associated with device 105. In some examples, application 140 may include one or more software widgets. In some cases, application 140 may include source code to operate one or more of the systems or system components described below with reference to FIG. 2, or the method steps disclosed in FIGS. 3-7.

In some examples, device 105 may communicate with server 110 via network 115. Examples of network 115 may include any combination of cloud networks, local area networks (LAN), wide area networks (WAN), virtual private networks (VPN), wireless networks (using 805.11, for example), cellular networks (using 3G, LTE, or 5G, for example), etc. In some configurations, the network 115 may include the Internet. In some examples, the device 105 may not include portal manager 145. For example, device 105 may include application 140 that allows device 105 to interface with a separate device via portal manager 145 being located on another device such as a separate computing device, server 110, database 120, or any combination thereof.

In some examples, at least one of device 105, database 120, and server 110 may include portal manager 145 where at least a portion of the functions of portal manager 145 are performed separately or concurrently on device 105, database 120, and/or server 110. In some examples, a user may access the functions of device 105 (directly or through device 105 via portal manager 145) from database 120 or server 110. In some examples, database 120 includes a mobile application that interfaces with one or more functions of device 105, server 110, or portal manager 145.

In some examples, server 110 may be coupled to database 120. Database 120 may be internal or external to the server 110. In one example, device 105 may be coupled to database 120. In some examples, database 120 may be internally or externally connected directly to device 105. Additionally, or alternatively, database 120 may be internally or externally connected directly to computing device 150 or one or more network devices such as a gateway, switch, router, intrusion detection system, etc. Database 120 may include portal manager 145. In some examples, device 105 may access or operate aspects of portal manager 145 from database 120 over network 115 via server 110. Database 120 may include script code, hypertext markup language code, procedural computer programming code, compiled computer program code, object code, uncompiled computer program code, object-oriented program code, class-based programming code, cascading style sheets code, or any combination thereof.

In one example, device 105 may be coupled to database 120. In some examples, database 120 may be internally or externally connected directly to device 105. Additionally, or alternatively, database 120 may be internally or externally connected directly to one or more network devices such as a gateway, switch, router, intrusion detection system, etc.

Portal manager 145 may enable a variety of features and functionality related to the systems described herein. In some examples, portal manager 145 may be configured to perform the systems and methods described herein in conjunction with user interface 135 and application 140. User interface 135 may enable a user to interact with, control, or program one or more functions of portal manager 145.

In some examples, portal manager 145 enables discovery, accessibility, and/or delivery of healthcare services and healthcare providers associated with a particular facility that may be proximate the device 105 and/or the patient. In some examples, portal manager 145 may be part of or work in coordination with a custom app that is installed on a user's mobile communication device that detects the user's/patient's location. Once the user/patient has the custom app installed, the custom app may detect the user's/patient's location while using that mobile computing device with the app installed and make recommendations, provide directions, and/or automatically notify the user/patient of notifications. With the app functional on the user's mobile computing device (e.g., smart phone), the mobile computing device (via the app) will automatically notify the user/patient of scheduling changes.

Even without the app installed, accessing the portal may be relatively easy for a user by simply visiting a predetermined URL in the user's mobile browser. By simply searching for the portal or typing the predetermined URL into the browser it will display the portal if the user does not have the app installed on their mobile computing device or the mobile computing device will open the app and show them the portal.

The portal may also include a feedback loop allowing a training system to learn what procedures are relevant to a specific facility. The training may be part of a machine learning (ML) technique and may include artificial intelligence (AI). The training system may include one or more algorithms operable using the system, devices and/or methods disclosed herein.

FIG. 2 illustrates an example of a portion of a system architecture 200 that supports arranging, completing, and paying for healthcare services using a portal in accordance with aspects of the present disclosure. In some examples, system architecture 200 may implement aspects of portal system 100. In the illustrated example, system architecture 200 is an example of the underlying components, functions, and structure used to carry out one or more of the methods disclosed herein. The system 200 is exemplary only of some or all of the inventive aspects of the present disclosure. Other example systems and methods may include more or feature steps, components or other features than those illustrated in the other figures described herein.

As shown, system architecture 200 enables healthcare providers to: approve procedure price or create custom offer, propose procedure dates, mark procedures as completed, list cash prices for procedures, and/or receive immediate cash payment. The system architecture 200 also enables patients to: search for procedures, request procedures, approve pricing, and/or pull payment. The system architecture 200 also enables payors to: pull payment, approve procedure pricing, and/or set member/patient responsibility. The system architecture 200 also enables the portal to: oversee progress, accept payment, pay providers, and enable all parties to share one conversation including documents, questions, and status updates.

FIG. 3 shows a flow chart illustrating a method 300 for discovering, accessing, arranging, paying for, and delivering of healthcare services to patients in accordance with aspects of the present disclosure. The operations of method 300 may be implemented by a device or its components as described herein. For example, the operation of method 300 may be performed by a portal manager as described with reference to FIG. 1. The portal manager described with reference to FIG. 1 may operate to perform some or all of the functions associated with the system 200 described with reference to FIG. 2. In some examples, a device may execute a set of instructions to control the functional elements of the device to perform the functions described herein. Additionally, or alternatively, a device may perform aspects of the functions described herein using special purpose hardware.

The method 300 includes: requesting 302 a healthcare procedure, scheduling 304 the healthcare procedure, confirming 306 pricing for the healthcare procedure, authorizing 308 payment for the healthcare procedure, funding 310 the healthcare procedure, completing 312 the healthcare procedure, paying 314 for the healthcare procedure, and communicating 316 with the healthcare provider, the payor, and/or the facility using the portal.

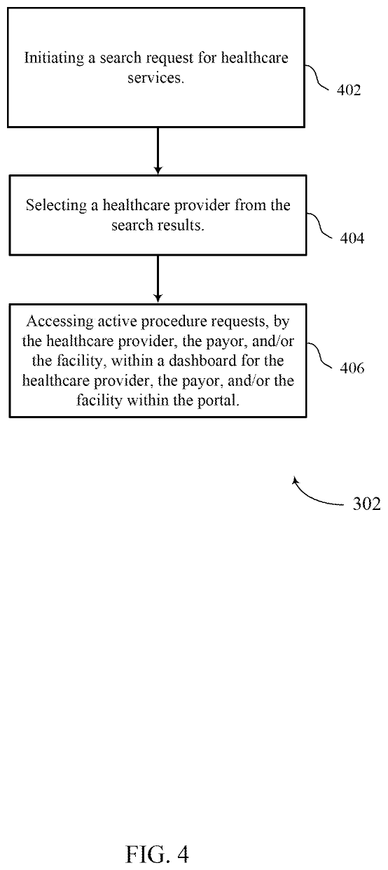

FIG. 4 shows a flow chart illustrating a method 302 for requesting a healthcare procedure in accordance with aspects of the present disclosure. Requesting 302 the healthcare procedure may include: (1) initiating 402 a search request for healthcare services, (2) selecting 404 a healthcare provider from the search results; and (3) accessing active procedure requests 406, by the healthcare provider, the payor, and/or the facility, within a dashboard for the healthcare provider, the payor, and/or the facility within the portal. Specifically, representatives of the payor's and/or members/patients log into the portal to initiate a search request for healthcare services. The search criteria may include a CPT code or procedure description, as well as a zip code and mile radius. The search results will display a cash price for the healthcare service and/or the facility and may also include other costs associated with the procedure, including the surgeon, anesthesiologist, physical therapist, or hardware and supplies.

When the representatives of the payors and/or members/patients selects a healthcare provider from the search results, the procedure request begins and prompts the user to share additional information about the member/patient, the member's/patient's physician, the procedure being requested, and the timeline for the procedure. The representatives of the payors and/or members/patients can access active procedure requests within a dashboard. Once the representatives of the payors and/or members/patients logs into the portal, access is granted to engage with healthcare providers to proceed with a procedure request. The member/patient must first sign a “HIPAA Consent and Release” document before proceeding with the procedure request.

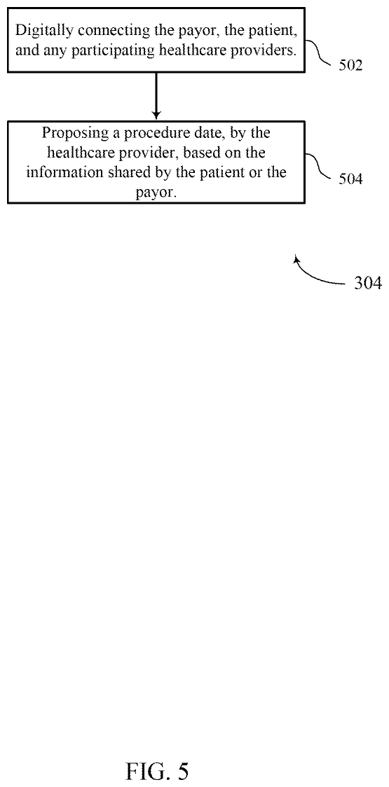

FIG. 5 shows a flow chart illustrating a method 304 for scheduling the healthcare procedure in accordance with aspects of the present disclosure. The method 304 includes: digitally connecting 502 the payor, the patient, and any participating healthcare providers; and proposing 504 a procedure date, by the healthcare provider, based on the information shared by the patient or the payor. The portal provides a digital connection between the payor, the patient, and any participating healthcare providers. Healthcare providers can include the facility (surgical center), the physician (surgeon), and other requested providers (physical therapist, anesthesiologist, etc.). Healthcare providers can be added to a specific procedure request by selecting from a list of active providers within the portal. Once added, the healthcare provider is notified of their addition to a procedure request. The portal also enables the healthcare provider to propose a procedure date, based on the information shared by the patient or the payor. This proposed procedure date can be accepted or declined by the patient. If declined, the healthcare provider will review the comments provided by the patient and propose an alternative procedure date for approval.

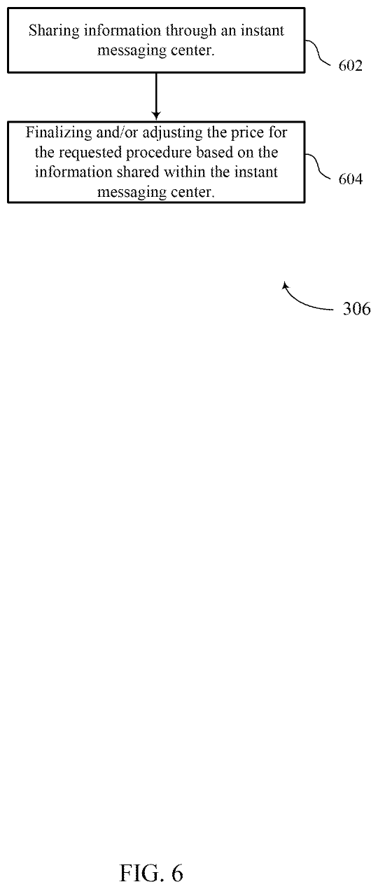

FIG. 6 shows a flow chart illustrating a method 306 for confirming pricing in accordance with aspects of the present disclosure. The method 306 includes: (1) sharing 602 information through an instant messaging center; and (2) finalizing 604 and/or adjusting the price for the requested procedure based on the information shared within the instant messaging center. Healthcare providers are given the ability to make adjustments to cash prices based on information shared in the portal. This flexibility afforded to providers is unmatched in other systems. Most other systems require providers to build a custom bundle, in advance, and offer that price. Other systems do not allow for flexibility or adjustments to pricing to accommodate circumstances. Also, providers are able to “finalize” or “lock” their price in the system, so the patient knows this is the final, agreed upon price. When all providers “lock” their price, they are agreeing to accept this price as payment in full for services rendered. The system generates a “Good Faith Estimate” in accordance with the No Surprise Billing Act to ensure providers are in compliance with the act, and to give legal documentation to patients regarding the price offered.

Once all stakeholders are connected within the portal, information is shared and communicated through the instant messaging center. This allows all participating stakeholders to confirm what care is being delivered, share the necessary documentation, ensure the requested procedure is a covered benefit by the payor, and any other necessary conversations. Healthcare providers may finalize their price for the requested procedure, or to make any necessary adjustments to the price based on the information shared within the instant messaging center. Healthcare providers may build an unlimited number of unique combinations of procedure codes or participating providers in order to create a custom bundled price for the medical procedure request.

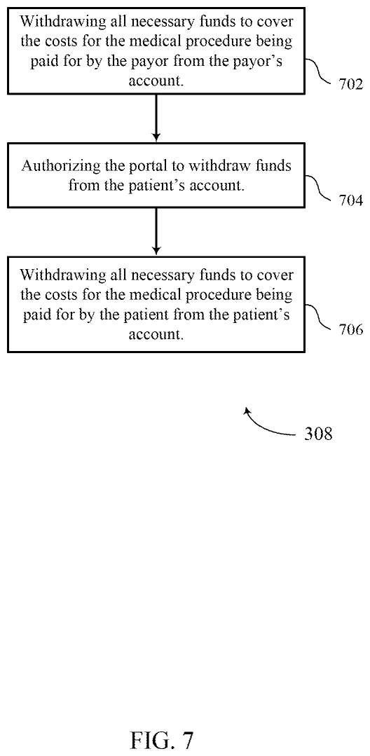

FIG. 7 shows a flow chart illustrating a method 308 for authorizing payment for the healthcare procedure in accordance with aspects of the present disclosure. The method 308 includes: (1) withdrawing 702 all necessary funds to cover the costs for the medical procedure being paid for by the payor from the payor's account; (2) authorizing 704 the portal to withdraw funds from the patient's account; and (3) withdrawing 706 all necessary funds to cover the costs for the medical procedure being paid for by the patient from the patient's account. The system connects patients/payors with affordable prices because the system ensures “Immediate Payment” to providers. Most providers deal with ambiguous contracts, lengthy receivable schedules, denied claims, collection headaches, and so many other complex issues trying to get their money for services rendered. These problems have created a whole new industry in healthcare called “Revenue Cycle Management”, basically selling providers tools to try and help them collect their money and get paid faster. The system eliminates all of these hassles by ensuring providers get paid exactly what they asked for, as soon as care is delivered.

The portal is a financial platform that ensures that healthcare providers are paid immediately and simplifies the payment process by patients and payors. The payor authorizes the portal to pull from the payor bank account all necessary funds to cover the costs for the medical procedure being paid for by the payor. This financial transaction requires the payor to link an active bank account to the portal and to have funds available for ACH withdrawal up to 5 business days prior to the scheduled procedure date. The patient authorizes the portal to collect any costs for the medical procedure that are the responsibility of the patient, including deductibles or other cost sharing obligations, or for services that are not covered by the payor. This financial transaction requires the patient to link an active bank account or credit card to the portal and to have funds available for withdrawal up to 5 business days prior to the scheduled procedure date.

Funding 310 the healthcare procedure may include depositing all funds from the payor and the patient 5 business days prior to the scheduled procedure date. Once the funds are deposited into a portal clearing account, the portal shows the Procedure Request as “Funded”. Healthcare providers can confirm the procedure is funded prior to delivering medical care to verify that payment will be made upon the completion of the medical procedure.

Completing 312 the healthcare procedure may include marking the procedure as complete within the portal. Once a Procedure Request has been completed, the healthcare provider will mark the procedure complete within the Portal. This step indicates the healthcare provider has delivered the agreed upon care and is ready to receive the agreed upon payment.

Paying 314 for the healthcare procedure may include releasing payment to all providers immediately upon completion of a medical procedure. The portal sends payment via ACH to every healthcare provider that provided an independent price as part of the Finalized Price within the Procedure Request.

Communicating 316 with the healthcare provider, the payor, and/or the facility using the portal may include facilitating communication between the healthcare provider, the payor, the patient, and/or the facility using an instant messaging center.

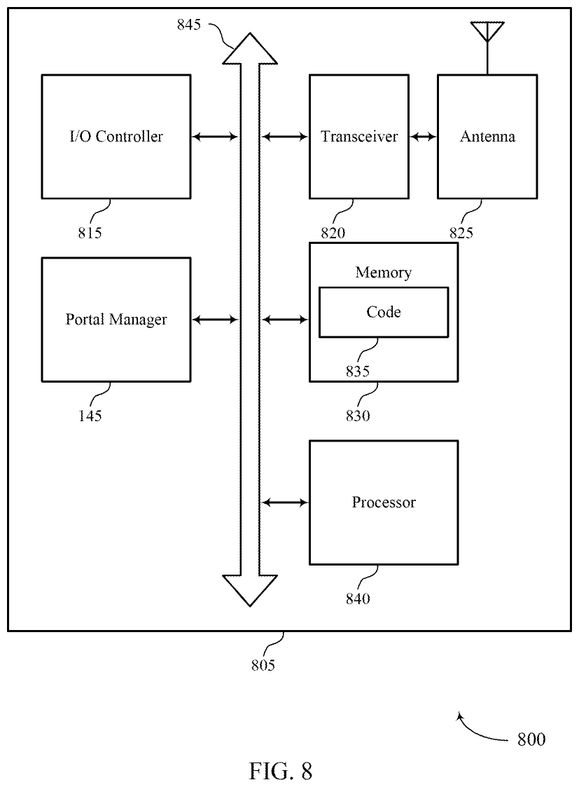

FIG. 8 shows a diagram of a system 800 including a device 805 that supports discovering, accessing, arranging, paying for, and delivering of healthcare services to patients in accordance with aspects of the present disclosure. The device 805 may be an example of or include the components of device 105 or devices as described herein. The device 805 may include components for bi-directional voice and data communications including components for transmitting and receiving communications, including a portal manager 145, an I/O controller 815, a transceiver 820, an antenna 825, memory 830, a processor 840, and a coding manager 835. These components may be in electronic communication via one or more buses.

The portal manager 145 may provide any combination of the operations and functions described above related to the system architecture 200 and the methods described herein.

The I/O controller 815 may manage input and output signals for the device 805. The I/O controller 815 may also manage peripherals not integrated into the device 805. In some cases, the I/O controller 815 may represent a physical connection or port to an external peripheral. In some cases, the I/O controller 815 may utilize an operating system such as iOS®, ANDROID®, MS-DOS®, MS-WINDOWS®, OS/2®, UNIX®, LINUX®, or another known operating system. In other cases, the I/O controller 815 may represent or interact with a modem, a keyboard, a mouse, a touchscreen, or a similar device. In some cases, the I/O controller 815 may be implemented as part of a processor. In some cases, a user may interact with the device 805 via the I/O controller 815 or via hardware components controlled by the I/O controller 815.

The transceiver 820 may communicate bi-directionally, via one or more antennas, wired, or wireless links as described herein. For example, the transceiver 820 may represent a wireless transceiver and may communicate bi-directionally with another wireless transceiver. The transceiver 820 may also include a modem to modulate the packets and provide the modulated packets to the antennas for transmission, and to demodulate packets received from the antennas.

In some cases, the wireless device may include a single antenna 825. However, in some cases the device may have more than one antenna 825, which may be capable of concurrently transmitting or receiving multiple wireless transmissions.

The memory 830 may include RAM and ROM. The memory 830 may store computer-readable, computer-executable code 835 including instructions that, when executed, cause the processor to perform various functions described herein. In some cases, the memory 830 may contain, among other things, a BIOS which may control basic hardware or software operation such as the interaction with peripheral components or devices.

The processor 840 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 840 may be configured to operate a memory array using a memory controller. In other cases, a memory controller may be integrated into the processor 840. The processor 840 may be configured to execute computer-readable instructions stored in a memory (e.g., the memory 830) to cause the device 805 to perform various functions (e.g., functions or tasks supporting related functions and other functions associated with the systems and methods disclosed herein).

The code 835 may include instructions to implement aspects of the present disclosure, including instructions to support dynamic accessibility compliance of a website. The code 835 may be stored in a non-transitory computer-readable medium such as system memory or other type of memory. In some cases, the code 835 may not be directly executable by the processor 840 but may cause a computer (e.g., when compiled and executed) to perform functions described herein.

It should be noted that the methods described herein describe possible implementations, and that the operations and the steps may be rearranged or otherwise modified and that other implementations are possible. Furthermore, aspects from two or more of the methods may be combined.

Techniques described herein may be used for various wireless communications systems such as code division multiple access (CDMA), time division multiple access (TDMA), frequency division multiple access (FDMA), orthogonal frequency division multiple access (OFDMA), single carrier frequency division multiple access (SC-FDMA), and other systems. The terms “system” and “network” are often used interchangeably. A code division multiple access (CDMA) system may implement a radio technology such as CDMA2000, Universal Terrestrial Radio Access (UTRA), etc. CDMA2000 covers IS-2000, IS-95, and IS-856 standards. IS-2000 Releases may be commonly referred to as CDMA2000 1×, 1×, etc. IS-856 (TIA-856) is commonly referred to as CDMA2000 1×EV-DO, High Rate Packet Data (HRPD), etc. UTRA includes Wideband CDMA (WCDMA) and other variants of CDMA. A time division multiple access (TDMA) system may implement a radio technology such as Global System for Mobile Communications (GSM). An orthogonal frequency division multiple access (OFDMA) system may implement a radio technology such as Ultra Mobile Broadband (UMB), Evolved UTRA (E-UTRA), IEEE 805.11 (Wi-Fi), IEEE 805.16 (WiMAX), IEEE 805.20, Flash-OFDM, etc.

The wireless communications system or systems described herein may support synchronous or asynchronous operation. For synchronous operation, the stations may have similar frame timing, and transmissions from different stations may be approximately aligned in time. For asynchronous operation, the stations may have different frame timing, and transmissions from different stations may not be aligned in time. The techniques described herein may be used for either synchronous or asynchronous operations.

The downlink transmissions described herein may also be called forward link transmissions while the uplink transmissions may also be called reverse link transmissions. Each communication link described herein—including, for example, portal system 100 and/or system architecture 200 of FIGS. 1 and 2, respectively—may include one or more carriers.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described herein may be implemented using software executed by a processor, hardware, firmware, hardwiring, or combinations of any of these. Features implementing functions may also be physically located at various positions, including being distributed such that portions of functions are implemented at different physical venues. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable read-only memory (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, digital subscriber line (DSL), or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method of arranging, completing, and paying for healthcare services using a portal, the method comprising:
requesting a healthcare procedure using the portal;
scheduling the healthcare procedure using the portal;
funding the healthcare procedure using the portal;
completing the healthcare procedure using the portal; and
paying for the healthcare procedure using the portal.

2. The method of claim 1 further comprising:
confirming pricing for the healthcare procedure using the portal.

3. The method of claim 2, wherein confirming pricing for the healthcare procedure using the portal comprises:
sharing information through an instant messaging center.

4. The method of claim 2, wherein confirming pricing for the healthcare procedure using the portal comprises:
adjusting a price for the requested procedure based on the information shared within the instant messaging center.

5. The method of claim 2, wherein confirming pricing for the healthcare procedure using the portal comprises:
finalizing a price for the requested procedure based on the information shared within the instant messaging center.

6. The method of claim 1 further comprising:
authorizing payment for the healthcare procedure using the portal.

7. The method of claim 6, wherein authorizing payment for the healthcare procedure using the portal comprises:
authorizing the portal to withdraw funds from a payor's account.

8. The method of claim 7, wherein authorizing payment for the healthcare procedure using the portal comprises:
withdrawing all necessary funds to cover the costs for the healthcare procedure being paid for by a payor from the payor's account.

9. The method of claim 6, wherein authorizing payment for the healthcare procedure using the portal comprises:
authorizing the portal to withdraw funds from a patient's account.

10. The method of claim 9, wherein authorizing payment for the healthcare procedure using the portal comprises:
withdrawing all necessary funds to cover the costs for the healthcare procedure being paid for by a patient from the patient's account.

11. The method of claim 1 further comprising:
communicating with a healthcare provider, a payor, a patient, and/or a facility using the portal.

12. The method of claim 11, wherein further communicating with the healthcare provider, the payor, the patient, and/or the facility using an instant messaging center.

13. The method of claim 1, wherein requesting a healthcare procedure using the portal comprises:
initiating a search request for healthcare services.

14. The method of claim 13, wherein requesting a healthcare procedure using the portal comprises:
selecting a healthcare provider from a set of search results.

15. The method of claim 14, wherein requesting a healthcare procedure using the portal comprises:
accessing active procedure requests.

16. The method of claim 15, wherein accessing active procedure requests comprises:
accessing active procedure requests, by a healthcare provider, a payor, and/or a facility, within a dashboard for the healthcare provider, the payor, and/or the facility within the portal.

17. The method of claim 1, wherein scheduling the healthcare procedure using the portal comprises:
digitally connecting a payor, a patient, and a healthcare provider.

18. The method of claim 17, wherein scheduling the healthcare procedure using the portal comprises:
proposing a procedure date, by the healthcare provider, based on the information shared by the patient or the payor.

19. The method of claim 1, wherein funding the healthcare procedure using the portal comprises depositing all funds from a payor and a patient into a portal clearing account.

20. The method of claim 1, wherein paying for the healthcare procedure using the portal comprises releasing payment to all healthcare providers immediately upon completion of the healthcare procedure.
